(19) United States
(12) Patent Application Publication
(10) Pub. No.: US 2014/0328774 A1
(43) Pub. Date: Nov. 6, 2014

(54) OIL-IN-WATER SKIN CARE COMPOSITION

(71) Applicant: THE PROCTER & GAMBLE COMPANY, Cincinnati, OH (US)

(72) Inventors: Suryamani ROUT, Singapore (SR); Naohisa YOSHIMI, Singapore (SR)

(73) Assignee: The Procter & Gamble Company, Cincinnati, OH (US)

(21) Appl. No.: 13/886,436

(22) Filed: May 3, 2013

(51) Int. Cl. A61K 8/06; A61Q 17/00

(52) U.S. Cl. CPC A61K 8/062; A61Q 17/00
USPC 424/59; 514/772.3

(57) ABSTRACT

An oil-in-water emulsion skin care composition comprising from about 3% to about 25%, by weight of the composition, of a hydrocarbon oil or a mixture thereof, from about 2% to about 15%, by weight of the composition, of silicone particles or a mixture thereof, from about 0.1% to about 5%, of an emulsifier or a mixture thereof, and from about 30% to about 90%, by weight of the composition, of water. The composition provides a pleasing skin sensory without much affecting occlusivity.

### FIELD OF THE INVENTION

The present invention relates to an oil-in-water emulsion skin care composition. The present invention also relates to a skin care method comprising applying the composition.

### BACKGROUND OF THE INVENTION

Occlusive agents, such as hydrocarbon oils, have been used in skin care products to deliver high occlusivity. Such high occlusivity is desired as it enhances the penetration into the skin of skin care actives contained in the skin care products. Specifically, occlusive agents form a film on the skin and therefore prevent moisture evaporation from the skin, resulting in an enhanced absorption of skin care actives, particularly water soluble actives, into the skin.

However, the incorporation of occlusive agents renders skin care products to have a negative skin sensory, e.g., a greasy, sticky feeling, and is thus disliked by users. By contrast, users prefer skin care products that provide a pleasing skin sensory, like a fresh feel or lower draginess after application.

Various approaches have been used to improve the skin sensory of skin care products. For instance, one approach is to add volatile oils. Volatile oils such as low viscosity cyclic silicone oils are commonly used in skin care products to deliver a fresh feel on contact with the skin due to their quick evaporation after being applied onto the skin. However, the incorporation of volatile oils typically leads to a significant decrease in occlusivity due to a diluted level of occlusive agents and thus a poor penetration of skin care actives.

An alternative approach is to use oil-in-water emulsions that contain no or limited amounts of occlusive agents. Such oil-in-water emulsions have a better skin sensory than water-in-oil emulsions that typically contain a high amount of occlusive agents. Nevertheless, on the one hand, the incorporation of no or limited amounts of occlusive agents leads to a poor penetration of skin care actives, as mentioned previously. On the other hand, it is undesirable to increase the amounts of occlusive agents in the oil-in-water emulsions, as it results in a worse skin sensory. In conclusion, there is an unmet need in current oil-in-water emulsions for skin care applications to achieve both pleasing skin sensory and sufficient penetration of skin care actives, i.e., higher occlusivity.

Thus, there is a need for an oil-in-water emulsion skin care composition that provides a pleasing skin sensory without much affecting occlusivity. It is the objective of the present invention to provide an oil-in-water emulsion skin care composition having a pleasing skin sensory as well as relatively high occlusivity.

It has been found that this objective can be met by the composition according to the present invention.

It is an advantage of the present invention to provide a skin care method that is efficient and less time consuming for users, i.e., providing a sufficient penetration of skin care actives within one step of applying a single composition onto skin, without requiring separate steps of applying two or more compositions onto skin in a particular sequence.

It is a further advantage of the present invention to provide an oil-in-water emulsion skin care composition that is aesthetically appealing to users.

### SUMMARY OF THE INVENTION

The present invention relates to an oil-in-water emulsion skin care composition, comprising: from about 3% to about 25%, by weight of the composition, of a hydrocarbon oil or a mixture thereof, from about 2% to about 15%, by weight of the composition, of silicone particles or a mixture thereof, from about 0.1% to about 5%, by weight of the composition, of an emulsifier or a mixture thereof, and from about 30% to about 90%, by weight of the composition, of water.

Preferably, the present invention relates to an oil-in-water emulsion skin care composition, comprising: from about 3% to about 25%, by weight of the composition, of a hydrocarbon oil or a mixture thereof, from about 2% to about 15%, by weight of the composition, of silicone particles or a mixture thereof, from about 0.1% to about 5%, by weight of the composition, of an emulsifier or a mixture thereof, from about 2% to about 15%, by weight of the composition, of a silicone oil or a mixture thereof, and from about 30% to about 90%, by weight of the composition, of water.

More preferably, the present invention relates to an oil-in-water emulsion skin care composition, comprising from about 5% to about 15%, by weight of the composition, of a hydrocarbon oil or a mixture thereof, from about 3% to about 10%, by weight of the composition, of silicone particles or a mixture thereof, from about 0.3% to about 1.5%, by weight of the composition, of an emulsifier or a mixture thereof, from about 3% to about 10%, by weight of the composition, of a silicone oil or a mixture thereof, and from about 30% to about 88%, by weight of the composition, of water.

Even more preferably, the present invention relates to an oil-in-water emulsion skin care composition, comprising: from about 7% to about 10%, by weight of the composition, of a hydrocarbon oil or a mixture thereof, from about 3% to about 5%, by weight of the composition, of silicone particles or a mixture thereof, from about 0.5% to about 1%, by weight of the composition, of an emulsifier or a mixture thereof, from about 3% to about 5%, by weight of the composition, of a silicone oil or a mixture thereof, and from about 50% to about 85%, by weight of the composition, of water.

The present invention further relates to a skin care method comprising applying the composition onto skin.

### DETAILED DESCRIPTION OF THE INVENTION

### Definitions

As used herein, the term “skin care product” means a product that is used to treat or care for, or somehow moisturize, improve, or clean the skin The skin care products include, but are not limited to skin moisturizers, skin cleansers, skin treatment emulsions, skin toners, skin remedial products, skin exfoliants, shaving creams, skin masks, and the like.

As used herein, the term “skin care composition” means a composition that constitutes the main part of a skin care product. The skin care composition generally comprises skin care actives, which deliver the function of treating, moisturizing, improving, or cleaning the skin.

As used herein, the term “oil-in-water emulsion” refers to an emulsion, in which oil is the dispersed liquid and an aqueous solution is the continuous phase.

As used herein, the term “water-in-oil emulsion” refers to an emulsion when water is the dispersed phase in the continuous oil phase.

As used herein, the term “occlusivity” refers to covering the skin with a film and thus preventing moisture evaporation from the skin. The term “occlusive agent” means a material that delivers the function of occlusivity when incorporated in a skin care product. The occlusive agents typically have relatively high occlusivity.

As used herein, the term “skin sensory” refers to the user perception for a skin care product that how the user feels about the product. In the present invention, criteria of fresh feel and draginess are used to characterize the skin sensory.

As used herein, the term “non-volatile” means a material that exhibits a vapor pressure of no more than 0.2 mm Hg at 25° C. at one atmosphere and/or a material that has a boiling point at one atmosphere of at least 300° C. The term “volatile”, as used herein, refers to all materials that are not “non-volatile” as defined herein.

As used herein, the articles including “a” and “an” when used in a claim, are understood to mean one or more of what is claimed or described.

As used herein, the terms “comprise”, “comprises”, “comprising”, “include”, “includes”, “including”, “contain”, “contains”, and “containing” are meant to be non-limiting, i.e., other steps and other ingredients which do not affect the end of result can be added. The above terms encompass the terms “consisting of and “consisting essentially of”.

### Skin Care Composition

The oil-in-water emulsion skin care composition of the present invention comprises from about 3% to about 25%, by weight of the composition, of a hydrocarbon oil or a mixture thereof, from about 2% to about 15%, by weight of the composition, of silicone particles or a mixture thereof, from about 0.1% to about 5%, by weight of the composition, of an emulsifier or a mixture thereof, from about 30% to about 90%, by weight of the composition, of water, and optionally an additional ingredient or a mixture thereof.

Preferably, the composition herein comprises from about 5% to about 15%, by weight of the composition, of the hydrocarbon oil or a mixture thereof, from about 3% to about 10%, by weight of the composition, of the silicone particles or a mixture thereof, from about 0.3% to about 1.5%, by weight of the composition, of the emulsifier or a mixture thereof, from about 30% to about 90%, by weight of the composition, of water, and optionally an additional ingredient or a mixture thereof. More preferably, the composition comprises from about 7% to about 10%, by weight of the composition, of the hydrocarbon oil or a mixture thereof, from about 3% to about 5%, by weight of the composition, of the silicone particles or a mixture thereof, from about 0.5% to about 1%, by weight of the composition, of the emulsifier or a mixture thereof, from about 50% to about 85%, by weight of the composition, of water, and optionally an additional ingredient or a mixture thereof.

Preferably, the composition herein comprises an additional ingredient or a mixture thereof. In a preferred embodiment, the additional ingredient is selected from the group consisting of a silicone oil, a skin care active, and mixtures thereof.

Preferably, the composition herein comprises one or more adjunct ingredients.

### Hydrocarbon Oil

The oil-in-water emulsion skin care composition of the present invention comprises a hydrocarbon oil or a mixture thereof in an amount of from about 3% to about 25%, by weight of the composition. Preferably, the hydrocarbon oil is present in the composition in an amount of from about 5% to about 15%, more preferably from about 7% to about 10%, by weight of the composition.

Preferred examples of the hydrocarbon oils include: petrolatum, mineral oil, micro-crystalline wax, polyalkene, paraffin, cerasin, ozokerite, polyethylene, perhydrosqualene, poly alpha olefin, hydrogenated polyisobutene, and mixtures thereof. The most preferred example of the hydrocarbon oils is petrolatum (e.g., Perfecta® Petrolatum available from Sonneborn, Inc).

Without wishing to be bound by theory, the hydrocarbon oils herein function as an occlusive agent in the composition. They are solid or semisolid at normal temperatures (25° C.). When applied onto skin, they form a film on the skin, prevent water from being vaporized from the skin, and therefore retain water at the surface of the skin Thus, the dissolved state of skin care actives, particularly water soluble agents, is kept, and the absorption thereof into the skin is enhanced.

In addition, compared with compositions that contain a high amount of occlusive agents, e.g., more than 50% of hydrocarbon oils, oil-in-water emulsions that contain limited amounts of occlusive agents, e.g., less than 25% of hydrocarbon oils, typically are aesthetically more appealing to users due to formulating oil-soluble actives and water-soluble actives in the same composition as well as decreased greasiness and heaviness.

### Silicone Particles

The oil-in-water emulsion skin care composition of the present invention comprises silicone particles or a mixture thereof in an amount of from about 2% to about 15%, by weight of the composition. Preferably, the silicone particles are present in the composition in an amount of from about 3% to about 10%, more preferably from about 3% to about 5%, by weight of the composition.

Preferably, the silicone particles suitable for use herein are selected from the group consisting of silicone resin particles, silicone rubber particles, and mixtures thereof. In a preferred embodiment, the silicone particles are selected from the group consisting of spherical silicone resin particles, spherical silicone rubber particles, and mixtures thereof. In another preferred embodiment, the silicone particles are a mixture of silicone resin particles and silicone rubber particles.

Preferably, the silicone particles herein are silicone resin particles. More preferably, the silicone particles are spherical silicone resin particles. Even more preferably, the silicone particles have an average particle size of from about 0.01 μm to about 5 μm, preferably from about 0.1 μm to about 4 μm, more preferably from about 1 μm to about 3 μm, even more preferably of being about 2 μm.

In a highly preferred embodiment, the silicone particles are polymethylsilsesquioxane (e.g., KMP-590 available from Shin-Etsu Chemical).

In the present invention, the applicant has surprisingly found that a combination of the silicone particles with a hydrocarbon oil in an oil-in-water emulsion skin care composition, within specific levels, achieves a pleasing skin sensory without much affecting occlusivity. Specifically, it has been found that such a combination, namely, from about 2% to about 15%, by weight of the composition, of the silicone particles and from about 3% to about 25%, by weight of the composition, of the hydrocarbon oil, achieves a pleasing skin sensory, particularly an improved fresh feel and decreased draginess, as well as relatively high occlusivity.

### Emulsifier

The oil-in-water emulsion skin care composition of the present invention comprises an emulsifier or a mixture thereof in an amount of from about 0.1% to about 5%, by weight of the composition. Preferably, the emulsifier is present in the composition in an amount of from about 0.1% to about 2%, more preferably from about 0.3% to about 1.5%, even more preferably from about 0.5% to about 1%, by weight of the composition. The emulsifier herein generally helps disperse and suspend the discontinuous phase within the continuous phase in the oil-in-water emulsion.

A wide variety of conventional emulsifiers is suitable for use herein. Preferred examples of the emulsifiers include: hydrophobically-modified crosslinked polyacrylate polymers and copolymers, polyacrylamide polymers and copolymers, and polyacryloyldimethyl taurates. More preferred examples of the emulsifiers include: acrylates/C10-30 alkyl acrylate crosspolymer having tradenames Pemulen™ TR-1, Pemulen™ TR-2 (all available from Lubrizol); acrylates/steareth-20 methacrylate copolymer with tradename ACRYSOL™ 22 (from Rohm and Hass); polyacrylamide with tradename SEPIGEL 305 (from Seppic).

In a highly preferred embodiment, the emulsifier used herein is acrylates/C10-30 alkyl acrylate crosspolymer (e.g., Pemulen™ TR-2 available from Lubrizol) present in an amount of from about 0.3% to about 1.5%, preferably from about 0.5% to about 1%, more preferably of being about 0.5%, by weight of the composition.

### Silicone Oil

The oil-in-water emulsion skin care composition of the present invention may include a silicone oil or a mixture thereof as an additional ingredient. Suitable levels of the silicone oil herein are in the range of from about 2% to about 15%, preferably from about 3% to about 10%, more preferably from about 3% to about 5%, by weight of the composition.

The silicone oils herein can be selected from any silicone oils well known in the art. The silicone oils are disclosed in U.S. Pat. No. 4,781,917 issued to Luebbe et al. on Nov. 1, 1988, herein incorporated by reference in its entirety. In one embodiment, the silicone oil herein is selected from the group consisting of a volatile silicone oil, a non-volatile silicone oil, and mixtures thereof.

In a preferred embodiment, the silicone oil herein is a volatile silicone oil. More preferably, the silicone oil herein is selected from the group consisting of a linear volatile silicone oil, a cyclic volatile silicone oil, and mixtures thereof. Linear volatile silicone oils generally have a viscosity of less than 5 centistokes at 25° C., whereas the cyclic silicone oils have viscosities of less than 10 centistokes at 25° C. Additionally, a description of various silicone materials is found in Todd et al., “Volatile Silicone Fluids for Cosmetics”, Cosmetics and Toiletries, 91:27-32 (1976), herein incorporated by reference in its entirety.

In a more preferred embodiment, the silicone oil herein is selected from the group consisting of volatile silicone oils corresponding to the formula:

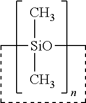

wherein n is from 3 to 7; linear volatile silicones corresponding to the formula:

(CH₃)₃ Si—O—Si(CH₃)₂—O]ₘ—Si(CH₃)₃

wherein m is from 1 to 7; and branched volatile silicones. Preferred examples of the volatile silicone oils include cyclomethicones of varying viscosities, e.g., Dow Corning° 200, Dow Corning° 244, Dow Corning® 245, Dow Corning® 344, and Dow Corning° 345, XIAMETER® PMX-0245 (all available from Dow Corning); SF-1204 and SF-1202 (all available from Momentive), GE 7207 and 7158 (all available from General Electric Co.); SWS-03314 (available from SWS Silicones); and KF-995 (available from Shin-Etsu Chemical). Preferred examples of the liner volatile silicone oils include KF-96A-1cs, KF-96L-1.5cs, KF-96L-2cs, KF-96A-5cs (all available from Shin-Etsu Chemical). Preferred examples of the branched volatile silicone oils include TMF-1.5 (available from Shin-Etsu Chemical). The most preferred example of the silicone oil is cyclopentasiloxane (e.g., SF-1202 available from Momentive).

Silicone oils, particularly volatile silicone oils, have been used in skin care compositions to deliver a fresh feel. However, the incorporation of such volatile silicone oils typically leads to a significant decrease in occlusivity and thus a poor penetration of skin care actives. In contrast, the applicant has surprisingly found that when combining the silicone oil with the hydrocarbon oil and silicone particles in an oil-in-water emulsion skin care composition, within specific levels, an excellent skin sensory is achieved without compromising occlusivity significantly. Specifically, it has been found that such a combination, namely, from about 3% to about 25%, by weight of the composition, of the hydrocarbon oil, from about 2% to about 15%, by weight of the composition, of the silicone particles, and from about 2% to about 15%, by weight of the composition, of the silicone oil, achieves an excellent skin sensory, particularly a substantially improved fresh feel and decreased draginess, without compromising occlusivity significantly.

### Skin Care Actives

A skin care active or a mixture thereof may be present in the oil-in-water emulsion skin care composition of the present invention as an additional ingredient and/or in a preparation that is applied onto skin prior to applying the composition of the present invention. In an embodiment, the skin care actives are present in the preparation that is applied onto skin prior to applying the composition of the present invention. In a preferred embodiment, the skin care actives are present in the composition of the present invention. Without wishing to be bound by theory, it is believed that either the present composition or the preparation that is applied onto skin prior to applying the present composition provides versatility in formulating a variety of skin care actives.

The skin care actives for use herein can be any skin care actives that deliver the function of treating, moisturizing, improving, or cleaning the skin. In one embodiment, the skin care actives herein can be classified by the benefit they provide or by their postulated mode of action. However, it is to be understood that the skin care actives herein in some instances provide more than one benefit or operate via more than one mode of action. Therefore, classifications herein are made for the sake of convenience and are not intended to limit the skin care actives to that particular application or applications listed.

In a preferred embodiment, the skin care actives herein are selected from the group consisting of a vitamin B3 compound, a whitening agent, a peptide, a sugar amine, a skin conditioning agent, a sunscreen agent, and mixtures thereof.

Vitamin B₃ Compound

Vitamin B3 compound such as niacinamide is a preferred skin care active for use herein. When used in the present composition or the preparation that is applied onto skin prior to applying the present composition, the vitamin B3 compound is present from about 0.1% to about 30%, preferably from about 1% to about 20%, more preferably from about 2% to about 10%, by weight of the composition or the preparation.

As used herein, the term “vitamin B3 compound” means a compound having the formula:

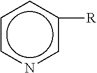

wherein R is —CONH₂ (i.e., niacinamide), —COOH (i.e., nicotinic acid) or —CH₂OH (i.e., nicotinyl alcohol); derivatives thereof; and salts of any of the foregoing. Exemplary derivatives of the foregoing vitamin B3 compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

Whitening Agent

A whitening agent may be included in the present composition and/or the preparation that is applied onto skin prior to applying the present composition. As used herein, the term “whitening agent” refers to active ingredients that not only alter the appearance of the skin, but further improve hyperpigmentation as compared to pre-treatment. When used in the present composition or the preparation that is applied onto skin prior to applying the present composition, the whitening agent is present from about 0.1% to about 10%, preferably from about 0.2% to about 5%, by weight of the composition or the preparation.

In one embodiment, the whitening agent herein is selected from the group consisting of ascorbic acid compound, azelaic acid, butyl hydroxy anisole, gallic acid and its derivatives, hydroquinoine, kojic acid, arbutin, mulberry extract, undecylenoyl phenylalanine, cetyl pyridinium chloride, glycyrrhizic acid, tetrahydrocurcumin, and mixtures thereof.

Preferred examples of the whitening agent are ascorbic acid compounds having the formula:

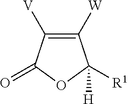

wherein V and W are independently —OH; R¹ is —CH(OH)—CH₂OH; and salts thereof. More preferably, the ascorbic acid compound herein is an ascorbic acid salt or derivative thereof, such as the non-toxic alkali metal, alkaline earth metal, and ammonium salts well known in the art including, but not limited to, the sodium, potassium, lithium, calcium, magnesium, barium, ammonium and protamine salts which are prepared by methods well known in the art.

Peptide

Peptides, including but not limited to, di-, tri-, tetra-, and pentapeptides and derivatives thereof, may be included in the present composition and/or the preparation that is applied onto skin prior to applying the present composition. As used herein, the term “peptides” refers to both the naturally occurring peptides and synthesized peptides. Also useful herein are naturally occurring and commercially available compositions that contain peptides.

When used in the present composition or the preparation that is applied onto skin prior to applying the present composition, a peptide is present in amounts that are safe and effective, preferably from about 1×10⁻⁶% to about 10%, more preferably from about 1×10⁻⁶% to about 0.1%, even more preferably from about 1×10⁻⁵% to about 0.01%, by weight of the composition or the preparation.

Sugar Amine

A sugar amine may be included in the present composition and/or the preparation that is applied onto skin prior to applying the present composition. As used herein, the term “sugar amine” refers to an amine derivative of a six-carbon sugar.

Preferred examples of the sugar amine herein include glucosamine, N-acetyl glucosamine, mannosamine, N-acetyl mannosamine, galactosamine, N-acetyl galactosamine. A more preferred example of the sugar amine is glucosamine. Additionally, combinations of two or more sugar amines may be used.

When used in the present composition or the preparation that is applied onto skin prior to applying the present composition, the sugar amine herein is present from about 0.001% to about 20%, preferably from about 1% to about 10%, more preferably from about 2% to about 5%, by weight of the composition or the preparation.

Skin Conditioning Agent

A skin conditioning agent may be included in the present composition and/or the preparation that is applied onto skin prior to applying the present composition. Preferably, such a skin conditioning agent is selected from the group consisting of humectants, exfoliants, emollients, and mixtures thereof.

Humectants are polyhydric alcohols intended for moisturizing, reducing scaling and stimulating removal of built-up scale from the skin. Typical polyhydric alcohols include polyalkylene glycols and more preferably alkylene polyols and their derivatives. Preferred examples of the humectants are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. The most preferred humectant is glycerin.

Exfoliants may be selected from the group consisting of C2-C30 alpha-hydroxycarboxylic acids, beta-hydroxycarboxylic acids, salts of these acids, and mixtures thereof. Preferred examples of the exfoliants are glycolic, lactic and salicylic acids and their ammonium salts. Emollients may be selected from the group consisting of hydrocarbons, fatty acids, fatty alcohols, esters, and mixtures thereof.

When used in the present composition or the preparation that is applied onto skin prior to applying the present composition, the skin-condition agent is present from about 1% to about 60%, preferably from about 2% to about 50%, more preferably from about 5% to about 40%, by weight of the composition or the preparation.

Sunscreen Agent

A sunscreen agent may be included in the present composition and/or the preparation that is applied onto skin prior to applying the present composition. As used herein, the term “sunscreen agent” refers to those that generally prevent excessive scaling and texture changes of the stratum corneum by exposure of ultraviolet light.

A wide variety of conventional sunscreen agents is suitable for use herein. Preferred examples of the sunscreen agents include octyl methoxylcinnamate, octyl salicylate, octocrylene, avobenzone, homosalate, octyl triazone, and mixtures thereof. Other conventional Sunscreen agents are also useful herein. Such agents include, for example, butylmethoxydibenzoyl-methane, 2-hydroxy-4-methoxybenzo-phenone, 2-phenylbenzimidazole-5-sulfonic acid, octyldimethyl-p-aminobenzoic acid, 2-ethylhexyl N,N-dimethyl-p-aminobenzoate, p-aminobenzoic acid, oxybenzone, 4-isopropyl dibenzoylmethane, 3-benzylidene camphor, 3-(4-methylbenzylidene)camphor.

When used in the present composition or the preparation that is applied onto skin prior to applying the present composition, the sunscreen agent is present from about 0.1% to about 20%, preferably from about 0.5% to about 10%, more preferably from about 1% to about 5%, by weight of the composition or the preparation. Exact amounts will vary depending upon the sunscreen agent chosen and the desired Sun Protection Factor (SPF). SPF is a commonly used measure of photoprotection of a sunscreen against erythema. See Federal Register, Vol. 43, No. 166, pp. 38206-38269, Aug. 25, 1978.

### Adjunct Ingredients

A variety of adjunct ingredients may be incorporated into the composition of the present invention. Preferably, the adjunct ingredients herein are selected from the group consisting of skin appearance modifying actives; anti-acne actives; oil-soluble vitamin compounds, terpene alcohols, phytosterol, beta-hydroxy acids such as salicylic acid, and derivatives thereof; chelators; flavonoid compounds; anti-inflammatory agents; anti-cellulite agents; desquamation actives; anti-oxidant/radical scavengers; tanning actives; skin soothing or skin healing actives such as panthenoic acid derivatives (including panthenol, dexpanthenol, ethyl panthenol), aloe vera, allantoin, bisabolol, and dipotassium glycyrrhizinate; antimicrobial or antifungal actives; and mixtures thereof.

### Composition Preparation

The oil-in-water emulsion skin care composition of the present invention is generally prepared by conventional methods such as are known in the art of making skin care compositions. Such methods typically involve mixing of the ingredients in one or more steps to a relatively uniform state, with or without heating, cooling, application of vacuum, and the like. Non-limiting examples of the product form can be a gel, emulsion, lotion, cream, ointment, solution, liquid, etc.

The composition herein may be prepared by the following steps:
a) heating a combination of a hydrocarbon oil and silicone particles at 70° C.-75° C., and then mixing the combination with an emulsifier by applying a shear of from 1000 rpm to 3000 rpm;
b) mixing the combination obtained in step a) with water at 70° C.-75° C. by applying a shear of 1000 rpm to 3000 rpm; and
c) homogenizing the combination obtained in step b) in a homogenizer at 8000 rpm to 12000 rpm for 8 minutes to 12 minutes, thus reducing the particle size of the combination and forming an oil-in-water emulsion composition,

wherein in the composition, the hydrocarbon oil is present in an amount of from about 3% to about 25%, the silicone particles are present in an amount of from about 2% to about 15%, the surfactant is present in an amount of from about 0.1% to about 5%, and water is present in an amount of from about 30% to about 90%, by weight of the composition.

In a preferred embodiment, the composition herein is prepared by the following steps:
a) heating a combination of a hydrocarbon oil and silicone particles at 70° C.-75° C., and then mixing the combination with an emulsifier by applying a shear of from 1000 rpm to 3000 rpm;
b) mixing the combination obtained in step a) with water at 70° C.-75° C. by applying a shear of 1000 rpm to 3000 rpm;
c) homogenizing the combination obtained in step b) in a homogenizer at 8000 rpm to 12000 rpm for 8 minutes to 12 minutes, thus reducing the particle size of the combination; and
d) once the temperature of the combination obtained in step c) is below 35° C., adding a silicone oil into the combination and mixing, and then homogenizing the mixture in the homogenizer at 8000 rpm to 12000 rpm for 8 minutes to 12 minutes, thus forming an oil-in-water emulsion composition,

wherein in the composition, the hydrocarbon oil is present in an amount of from about 3% to about 25%, the silicone particles are present in an amount of from about 2% to about 15%, the surfactant is present in an amount of from about 0.1% to about 5%, the silicone oil is present in an amount of from about 2% to about 15%, and water is present in an amount of from about 30% to about 90%, by weight of the composition.

In a more preferred embodiment, the composition herein is prepared by the following steps:
a) heating a combination of a hydrocarbon oil and silicone particles at 70° C.-75° C., and then mixing the combination with an emulsifier by applying a shear of from 1000 rpm to 3000 rpm;
b) mixing the combination obtained in step a) with water at 70° C.-75° C. by applying a shear of 1000 rpm to 3000 rpm;
c) homogenizing the combination obtained in step b) in a homogenizer at 8000 rpm to 12000 rpm for 8 minutes to 12 minutes, thus reducing the particle size of the combination and forming an oil-in-water emulsion;
d) once the temperature of the combination obtained in step c) is below 35° C., adding a silicone oil and a vitamin B3 compound into the combination and mixing, and then homogenizing the mixture in the homogenizer at 8000 rpm to 12000 rpm for 8 minutes to 12 minutes, thus forming an oil-in-water emulsion composition,

wherein in the composition, the hydrocarbon oil is present in an amount of from about 3% to about 25%, the silicone particles are present in an amount of from about 2% to about 15%, the surfactant is present in an amount of from about 0.1% to about 5%, the silicone oil is present in an amount of from about 2% to about 15%, the vitamin B3 compound is present in an amount of from about 1% to about 20%, and water is present in an amount of from about 30% to about 90%, by weight of the composition.

### Skin Care Method

The skin care method of the present invention comprises applying an oil-in-water emulsion skin care composition onto skin, wherein the composition comprises from about 3% to about 25%, by weight of the composition, of a hydrocarbon oil or a mixture thereof, from about 2% to about 15%, by weight of the composition, of silicone particles or a mixture thereof, from about 0.1% to about 5%, by weight of the composition, of an emulsifier or a mixture thereof; from about 30% to about 90%, by weight of the composition, of water, and optionally an additional ingredient or a mixture thereof.

In one embodiment, the skin care method herein is a multiple-step method, e.g., a two-step method. As used herein, the term “multiple-step” refers to requiring separate steps of applying two or more compositions onto skin in a particular sequence. Preferably, the skin care method comprises the steps of a) applying a preparation comprising a skin care active onto skin; and b) applying the composition of the present invention onto skin. More preferably, the skin care active herein is selected from the group consisting of a vitamin B3 compound, a whitening agent, a peptide, a sugar amine, a skin conditioning agent, a sunscreen agent, and mixtures thereof.

In another embodiment, the skin care method herein is a one-step method. As used herein, the term “one-step” refers to applying a single composition onto skin, without requiring separate steps of applying two or more compositions onto skin in a particular sequence. Preferably, the skin care method comprises applying a composition onto skin, wherein the composition comprises from about 3% to about 25%, by weight of the composition, of a hydrocarbon oil or a mixture thereof, from about 2% to about 15%, by weight of the composition, of silicone particles or a mixture thereof, from about 0.1% to about 5%, by weight of the composition, of an emulsifier or a mixture thereof, from about 30% to about 90%, by weight of the composition, of water, and a skin care active or a mixture thereof. More preferably, the skin care active is selected from the group consisting of a vitamin B3 compound, a whitening agent, a peptide, a sugar amine, a skin conditioning agent, a sunscreen agent, and mixtures thereof.

In the art, the multiple-step skin care method is generally considered more effective for skin care actives to penetrate into the skin, even though it is more time-consuming than the one-step method. Typically, the multiple-step method comprises the steps of applying a first composition comprising skin care actives onto the skin and then applying a second composition that contains occlusive agents and has high occlusivity onto the skin, thus ensuring a sufficient penetration of the skin care actives into the skin. However, in the present invention, the applicant has surprisingly found that the oil-in-water emulsion skin care composition, which comprises from about 3% to about 25%, by weight of the composition, of a hydrocarbon oil or a mixture thereof, from about 2% to about 15%, by weight of the composition, of silicone particles or a mixture thereof, from about 0.1% to about 5%, by weight of the composition, of an emulsifier or a mixture thereof, from about 30% to about 90%, by weight of the composition, of water or a mixture thereof, and a skin care active or a mixture thereof, provides a relatively high occlusivity and therefore an enhanced penetration into the skin of the skin care active contained in the composition, without requiring a separate step of applying other compositions.

### Test Methods

### I. Method for Determining of Occlusivity for Skin Care Compositions

The method uses a standardized stainless-steel testing chamber into which a collagen film (Naturin COFFI J) is placed as semi-permeable membrane. The cup is filled Calcium chloride (10 g), and the material (0.535 g) to be tested is coated to on the rough surface of the membrane. The testing chamber is closed and placed in a temperature-controlled room initially at 25° C./60% RH room for 1 hour and then at 40° C./75% RH room for 24 hours. After the testing period the weight of the cup is measured. The weight of the cup is also measured after the removal of Chloride. The absorption of water vapor by the CaCl₂ is used for the calculation of occlusivity.

The occlusivity is calculated with the formula below:

Occlusivity (%)=100−((amount of total cup weight−amount of the cup w/o CaCl₂)−10 (Original CaCl₂)*10.0)

### II. Method for Determining of Skin Sensory for Skin Care Compositions

A total number of 10 qualified panelists have participated in the study. The forearms of the volunteers were washed soap and rinsed well with water, wiped with paper towel to dry out completely. 0.1 ml of test product and the Baseline were applied on forearms. The attributes of test product versus the Baseline were evaluated with dipolar scales (from −3 to +3). “0” indicates to be no difference compared with control. The results are the mean value of 10 volunteers participated in the study.

“Fresh feel” refers to the attribute that is not sticky and referred as a positive attribute. A positive value of the fresh feel means a better fresh feel than the Baseline, and a greater absolute value thereof means a more significant difference versus the Baseline.

“Draginess” refers to the resistance that the product creates while being applied onto the skin. A negative value of the draginess means better draginess than the Baseline, and a greater absolute value thereof means a more significant difference versus the Baseline.

### EXAMPLES

The Examples herein are meant to exemplify the present invention but are not used to limit or otherwise define the scope of the present invention. Examples 1A-1I are examples according to the present inventions, and Examples 2A-2D are comparative examples.

### Example 1A-1I

### Formulations of Oil-in-Water Emulsion Skin Care Compositions

The following compositions are made comprising the listed ingredients in the listed proportions (weight %).

TABLE 1

| | 1A | 1B | 1C | 1D | 1E | 1F | 1G | 1H | 1I |
|---|---|---|---|---|---|---|---|---|---|
| Perfecta ® | 7.5% | 7.5% | 15% | 7.5% | 7.5% | 20% | 15% | 15% | 5% |
| petrolatum a | | | | | | | | | |
| KMP-590 b | 7.5% | 7.5% | 7.5% | 3.75% | 3.75% | 10% | 7.5% | 5% | 5% |
| SF-1202 c | 0 | 0 | 0 | 3.75% | 3.75% | 10% | 7.5% | 5% | 5% |
| Pemulen ™ TR-2 d | 0.5% | 0.5% | 0.5% | 0.5% | 0.5% | 0.5% | 0.5% | 0.5% | 0.5% |
| niacinamide | 0 | 5% | 0 | 0 | 5% | 0 | 0 | 0 | 0 |
| Water | Add to | Add to | Add to | Add to | Add to | Add to | Add to | Add to | Add to |
| | 100% | 100% | 100% | 100% | 100% | 100% | 100% | 100% | 100% |

a Perfecta ® Petrolatum is available from Sonneborn, Inc
b KMP-590 is polymethylsilsesquioxane available from Shin-Etsu Chemical
c SF-1202 is cyclopentasiloxane available from Momentive
d Pemulen ™ TR-2 is acrylates/C10-30 alkyl acrylate crosspolymer available from Lubrizol

### Preparation of the Composition of Example 1A

The composition of Example 1A is prepared by the following steps:
a) heating a combination of Perfecta® Petrolatum and KMP-590 at 70° C., and then mixing the combination with Pemulen™ TR-2 by applying a shear of 2000 rpm;
b) mixing the combination obtained in step a) with water at 70° C. by applying a shear of 2000 rpm; and
c) homogenizing the combination obtained in step b) in TK Robomics Homogenizer at 10000 rpm for 10 minutes, thus reducing the particle size of the combination and forming an oil-in-water emulsion composition,

wherein in the composition, Perfecta® Petrolatum is present in an amount of 7.5%, KMP-590 is present in an amount of 7.5%, Pemulen™ TR-2 is present in an amount of 0.5%, and water is present in an amount of 84.5%, by weight of the composition.

### Preparation of the Composition of Example 1B

The composition of Example 1B is prepared by the following steps:
a) heating a combination of Perfecta® Petrolatum and KMP-590 at 70° C., and then mixing the combination with Pemulen™ TR-2 by applying a shear of 2000 rpm;
b) mixing the combination obtained in step a) with water at 70° C. by applying a shear of 2000 rpm;
c) homogenizing the combination obtained in step b) in TK Robomics Homogenizer at 10000 rpm for 10 minutes, thus reducing the particle size of the combination; and
d) once the temperature of the combination obtained in step c) is below 35° C., adding niacinamide into the combination and mixing, and then homogenizing the mixture in TK Robomics Homogenizer at 10000 rpm for 10 minutes, thus forming an oil-in-water emulsion composition,

wherein in the composition, Perfecta® Petrolatum is present in an amount of 7.5%, KMP-590 is present in an amount of 7.5%, Pemulen™ TR-2 is present in an amount of 0.5%, niacinamide is present in an amount of 5%, and water is present in an amount of 79.5%, by weight of the composition.

### Preparation of the Composition of Example 1C

The composition of Example 1C is prepared by the same steps as in Example 1A, except for the following: Perfecta® Petrolatum is present in an amount of 15%, and water is present in an amount of 77%, by weight of the composition.

### Preparation of the Composition of Example 1D

The composition of Example 1D is prepared by the following steps:
a) heating a combination of Perfecta® Petrolatum and KMP-590 at 70° C., and then mixing the combination with Pemulen™ TR-2 by applying a shear of 2000 rpm;
b) mixing the combination obtained in step a) with water at 70° C. by applying a shear of 2000 rpm;
c) homogenizing the combination obtained in step b) in TK Robomics Homogenizer at 10000 rpm for 10 minutes, thus reducing the particle size of the combination; and
d) once the temperature of the combination obtained in step c) is below 35° C., adding SF-1202 into the combination and mixing, and then homogenizing the mixture in TK Robomics Homogenizer at 10000 rpm for 10 minutes, thus forming an oil-in-water emulsion composition,

wherein in the composition, Perfecta® Petrolatum is present in an amount of 7.5%, KMP-590 is present in an amount of 3.75%, Pemulen™ TR-2 is present in an amount of 0.5%, SF-1202 is present in an amount of 3.75%, and water is present in an amount of 84.5%, by weight of the composition.

### Preparation of the Composition of Example 1E

The composition of Example 1E is prepared by the following steps:
a) heating a combination of Perfecta® Petrolatum and KMP-590 at 70° C., and then mixing the combination with Pemulen™ TR-2 by applying a shear of 2000 rpm;
b) mixing the combination obtained in step a) with water at 70° C. by applying a shear of 2000 rpm;
c) homogenizing the combination obtained in step b) in TK Robomics Homogenizer at 10000 rpm for 10 minutes, thus reducing the particle size of the combination; and
d) once the temperature of the combination obtained in step c) is below 35° C., adding SF-1202 and niacinamide into the combination and mixing, and then homogenizing the mixture in TK Robomics Homogenizer at 10000 rpm for 10 minutes, thus forming an oil-in-water emulsion composition,

wherein in the composition, Perfecta® Petrolatum is present in an amount of 7.5%, KMP-590 is present in an amount of 3.75%, Pemulen™ TR-2 is present in an amount of 0.5%, SF-1202 is present in an amount of 3.75%, niacinamide is present in an amount of 5%, and water is present in an amount of 79.5%, by weight of the composition.

### Preparation of the Composition of Example 1F

The composition of Example 1F is prepared by the same steps as in Example 1D, except for the following: Perfecta® Petrolatum is present in an amount of 20%, KMP-590 is present in an amount of 10%, Pemulen™ TR-2 is present in an amount of 0.5%, SF-1202 is present in an amount of 10%, and water is present in an amount of 59.5%, by weight of the composition.

### Preparation of the Composition of Example 1G

The composition of Example 1G is prepared by the same steps as in Example 1D, except for the following: Perfecta® Petrolatum is present in an amount of 15%, KMP-590 is present in an amount of 7.5%, Pemulen™ TR-2 is present in an amount of 0.5%, SF-1202 is present in an amount of 7.5%, and water is present in an amount of 69.5%, by weight of the composition.

### Preparation of the Composition of Example 1H

The composition of Example 1H is prepared by the same steps as in Example 1D, except for the following: Perfecta® Petrolatum is present in an amount of 15%, KMP-590 is present in an amount of 5%, Pemulen™ TR-2 is present in an amount of 0.5%, SF-1202 is present in an amount of 5%, and water is present in an amount of 74.5%, by weight of the composition.

### Preparation of the Composition of Example 11

The composition of Example 11 is prepared by the same steps as in Example 1D, except for the following: Perfecta® Petrolatum is present in an amount of 5%, KMP-590 is present in an amount of 5%, Pemulen™ TR-2 is present in an amount of 0.5%, SF-1202 is present in an amount of 5%, and water is present in an amount of 84.5%, by weight of the composition.

### Comparative Example 2A-2D

### Comparative Formulations of Oil-in-Water Emulsion Skin Care Compositions

The following comparative compositions are made comprising the listed ingredients in the listed proportions (weight %).

TABLE 2

| | 2A | 2B | 2C | 2D |
|---|---|---|---|---|
| Perfecta ® Petro- | 10% | 7.5% | 30% | 2% |
| latum a | | | | |
| KMP-590 b | 0 | 0 | 15% | 1.5% |
| SF-1202 c | 0 | 7.5% | 15% | 1.5% |
| Pemulen ™ TR-2 d | 0.5% | 0.5% | 0.5% | 0.5% |
| Water | Add to | Add to | Add to | Add to |
| | 100% | 100% | 100% | 100% |

a Perfecta ® Petrolatum is available from Sonneborn, Inc
b KMP-590 is polymethylsilsesquioxane available from Shin-Etsu Chemical
c SF-1202 is cyclopentasiloxane available from Momentive
d Pemulen ™ TR-2 is acrylates/C10-30 alkyl acrylate crosspolymer available from Lubrizol

### Preparation of the Composition of Comparative Example 2A

The composition of Example 2A is prepared by the following steps:
a) heating Perfecta® Petrolatum at 70° C., and then mixing it with Pemulen™ TR-2 by applying a shear of 2000 rpm;
b) mixing the combination obtained in step a) with water at 70° C. by applying a shear of 2000 rpm; and
c) homogenizing the combination obtained in step b) in TK Robomics Homogenizer at 10000 rpm for 10 minutes, thus reducing the particle size of the combination and forming an oil-in-water emulsion composition,

wherein in the composition, Perfecta® Petrolatum is present in an amount of 10%, Pemulen™ TR-2 is present in an amount of 0.5%, and water is present in an amount of 89.5%, by weight of the composition.

### Preparation of the Composition of Comparative Example 2B

The composition of Example 2B is prepared by the following steps:
a) heating Perfecta® Petrolatum at 70° C., and then mixing it with Pemulen™ TR-2 by applying a shear of 2000 rpm;
b) mixing the combination obtained in step a) with water at 70° C. by applying a shear of 2000 rpm;
c) homogenizing the combination obtained in step b) in TK Robomics Homogenizer at 10000 rpm for 10 minutes, thus reducing the particle size of the combination; and
d) once the temperature of the combination obtained in step c) is below 35° C., adding SF-1202 into the combination and mixing, and then homogenizing the mixture in TK Robomics Homogenizer at 10000 rpm for 10 minutes, thus forming an oil-in-water emulsion composition,

wherein in the composition, Perfecta® Petrolatum is present in an amount of 7.5%, Pemulen™ TR-2 is present in an amount of 0.5%, SF-1202 is present in an amount of 7.5%,and water is present in an amount of 84.5%, by weight of the composition.

### Preparation of the Composition of Comparative Example 2C

The composition of Example 2C is prepared by the following steps:
e) heating a combination of Perfecta® Petrolatum and KMP-590 at 70° C., and then mixing the combination with Pemulen™ TR-2 by applying a shear of 2000 rpm;
f) mixing the combination obtained in step a) with water at 70° C. by applying a shear of 2000 rpm;
g) homogenizing the combination obtained in step b) in TK Robomics Homogenizer at 10000 rpm for 10 minutes, thus reducing the particle size of the combination; and
h) once the temperature of the combination obtained in step c) is below 35° C., adding SF-1202 into the combination and mixing, and then homogenizing the mixture in TK Robomics Homogenizer at 10000 rpm for 10 minutes, thus forming an oil-in-water emulsion composition,

wherein in the composition, Perfecta® Petrolatum is present in an amount of 30%, KMP-590 is present in an amount of 15%, Pemulen™ TR-2 is present in an amount of 0.5%, SF-1202 is present in an amount of 15%, and water is present in an amount of 39.5%, by weight of the composition.

### Preparation of the Composition of Comparative Example 2D

The composition of Example 2D is prepared by the same steps as in Example 2C, except for the following: Perfecta® Petrolatum is present in an amount of 2%, KMP-590 is present in an amount of 1.5%, Pemulen™ TR-2 is present in an amount of 0.5%, SF-1202 is present in an amount of 1.5%, and water is present in an amount of 94.5%, by weight of the composition.

### Comparative Data of Examples 1 and 2

Comparative experiments of measuring the occlusivity and skin sensory of the compositions of Examples 1A, 1C, 1D, 1F to 1I, and 2A to 2D are conducted. The experimental results are shown in Table 3.

The occlusivity is measured according to the method for determining of occlusivity for skin care compositions as described herein above. It's worth noting that, an occlusivity value as low as 70% does not lead to a good penetration of niacinamide into the skin.

The skin sensory is measured according to the method for determining of skin sensory for skin care compositions as described herein above. Example 2A is taken as the Baseline for generating the data of fresh feel and draginess.

TABLE 3

| Example | Occlusivity (%) | Fresh Feel | Draginess |
|---|---|---|---|
| 2A | 90.3 | 0 | 0 |
| 1A | 88.2 | 0.6 | −1 |
| 1C | 96.5 | 0.66 | −0.77 |
| 1D | 77.0 | 1.2 | −1.4 |
| 1F | 95.6 | 0.3 | −0.4 |
| 1G | 92.0 | 0.66 | −0.88 |
| 1H | 90.2 | 0.3 | −0.4 |
| 1I | 78.0 | 0.9 | −1.1 |
| 2B | 75.1 | 0.7 | −0.7 |
| 2C | 96.5 | −0.2 | 0.5 |
| 2D | 70.5 | 0.11 | −0.22 |

As shown in Table 3, the oil-in-water emulsion skin care compositions according to the present invention (Examples 1A, 1C, 1D, 1F, 1G, 1H, and 1I) demonstrate a pleasing skin sensory without much affecting occlusivity, whereas the comparative compositions that are out of the scope of the present invention (Examples 2B, 2C, and 2D) show either a negative skin sensory or relatively low occlusivity. Specifically, Example 1A demonstrates a much higher value of occlusivity (88.2%) than Example 2B (75.1%), even though the amount of silicone particles in Example 1A is the same as that of silicone oils in Example 2B. Moreover, Example 2C demonstrates a quite negative skin sensory, namely, a low value of fresh feel (−0.2) and a high value of draginess (0.5) even though it has a quite high value of occlusivity (96.5%). By contrast, Example 1F achieves a value of occlusivity (95.6%) almost as high as Example 2C as well as a positive skin sensory, namely, a value of fresh feel at 0.3 and a value of draginess at −0.4. Furthermore, Example 2D demonstrates a quite low value of occlusivity (70.5%) and a skin sensory close to the Baseline. By contrast, Example 1I leads to an excellent skin sensory, namely, a value of fresh feel at 0.9 and a value of draginess at −1.1, whereas the value of occlusivity thereof (78.0%) is not too low.

Unless otherwise indicated, all percentages, ratios, and proportions are calculated based on weight of the total composition. All temperatures are in degrees Celsius (° C.) unless otherwise indicated. All measurements made are at 25° C., unless otherwise designated. All component or composition levels are in reference to the active level of that component or composition, and are exclusive of impurities, for example, residual solvents or by-products, which may be present in commercially available sources.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oil-in-water emulsion skin care composition, comprising:
from about 3% to about 25%, by weight of the composition, of a hydrocarbon oil or a mixture thereof;
from about 2% to about 15%, by weight of the composition, of silicone particles or a mixture thereof;
from about 0.1% to about 5%, by weight of the composition, of an emulsifier or a mixture thereof;
from about 30% to about 90%, by weight of the composition, of water; and
optionally an additional ingredient or a mixture thereof.

2. The composition according to claim 1, comprising from about 5% to about 15%, by weight of the composition, of said hydrocarbon oil.

3. The composition according to claim 2, comprising from about 7% to about 10%, by weight of the composition, of said hydrocarbon oil.

4. The composition according to claim 1, wherein said hydrocarbon oil is selected from the group consisting of petrolatum, mineral oil, micro-crystalline wax, polyalkene, paraffin, cerasin, ozokerite, polyethylene, perhydrosqualene, poly alpha olefin, hydrogenated polyisobutene, and mixtures thereof.

5. The composition according to claim 4, wherein said hydrocarbon oil is petrolatum.

6. The composition according to claim 1, comprising from about 3% to about 10%, by weight of the composition, of said silicone particles.

7. The composition according to claim 6, comprising from about 3% to about 5%, by weight of the composition, of said silicone particles.

8. The composition according to claim 1, wherein said silicone particles are selected from the group consisting of silicone resin particles, silicone rubber particles, and mixtures thereof.

9. The composition according to claim 1, wherein said emulsifier is selected from the group consisting of hydrophobically-modified crosslinked polyacrylate polymers and copolymers, polyacrylamide polymers and copolymers, polyacryloyldimethyl taurates, and mixtures thereof.

10. The composition according to claim 1, wherein said additional ingredient is a silicone oil, wherein said silicone oil is present in the composition from about 2% to about 15%.

11. The composition according to claim 10, comprising from about 3% to about 10%, by weight of the composition, of said silicone oil.

12. The composition according to claim 11, comprising from about 3% to about 5%, by weight of the composition, of said silicone oil.

13. The composition according to claim 1, comprising:
from about 7% to about 10%, by weight of the composition, of said hydrocarbon oil or a mixture thereof;
from about 3% to about 5%, by weight of the composition, of said silicone particles or a mixture thereof;
from about 0.5% to about 1%, by weight of the composition, of said emulsifier or a mixture thereof;
from about 3% to about 5%, by weight of the composition, of a silicone oil or a mixture thereof;
from about 50% to about 85%, by weight of the composition, of water; and
optionally an additional ingredient or a mixture thereof.

14. The composition according to claim 1, wherein said additional ingredient is a skin care active selected from the group consisting of a vitamin B3 compound, a whitening agent, a peptide, a sugar amine, a skin conditioning agent, a sunscreen agent, and mixtures thereof.

15. The composition according to claim 14, wherein said vitamin B3 compound is selected from the group consisting of niacinamide, derivatives of niacinamide, salts of niacinamide or derivatives of niacinamide, and mixtures thereof.

16. The composition according to claim 1, comprising an adjunct ingredient selected from the group consisting of skin appearance modifying actives; anti-acne actives; oil-soluble vitamin compounds, terpene alcohols, phytosterol, beta-hydroxy acids such as salicylic acid, and derivatives thereof; chelators; flavonoid compounds; anti-inflammatory agents; anti-cellulite agents; desquamation actives; anti-oxidant/radical scavengers; tanning actives; skin soothing or skin healing actives; antimicrobial or antifungal actives, and mixtures thereof.

17. A skin care method comprising applying the composition according to claim 1 onto skin

18. A skin care method comprising applying the composition according to claim 14 onto skin.

19. A skin care method comprising the steps of:
a) applying a preparation onto skin, wherein said preparation comprises a skin care active selected from the group consisting of a vitamin B3 compound, a whitening agent, a peptide, a sugar amine, a skin conditioning agent, a sunscreen agent, and mixtures thereof; and
b) applying the composition according to claim 1 onto skin.
